(19) United States
(12) Patent Application Publication
(10) Pub. No.: US 2009/0312558 A1
(43) Pub. Date: Dec. 17, 2009

(54) PROCESS FOR THE PREPARATION OF ARYLCYCLOPROPOANE CARBOXYLIC CARBONITRILES, AND COMPOUNDS DERIVED THEREFROM

(75) Inventors: Sandeep Pandurang Bhujbal, Maharashtra (IN); Venkata Ramana Kondepati, Maharashtra (IN); Rajesh Rao, Maharashtra (IN); Jayaraman Venkat Raman, Maharashtra (IN)

Correspondence Address: LACKENBACH SIEGEL, LLP, LACKENBACH SIEGEL BUILDING, 1 CHASE ROAD, SCARSDALE, NY, 10583, US

(73) Assignee: Reliance Life Sciences Pvt. Ltd., Navi Mumbai (IN)

(21) Appl. No.: 12/146,569

(22) Filed: Jun. 26, 2008

(30) Foreign Application Priority Data
Jun. 13, 2008 (IN) 1255/MUM/2008

(51) Int. Cl. C07D 249/08; C07C 255/01; C07C 61/04; C07C 33/00

(52) U.S. Cl. 548/267.8; 558/388; 562/405; 568/807

(57) ABSTRACT

The present invention relates to an efficient process for preparing an arylcyclopropanecarbonitrile, which involves the use of sulfolane as a solvent.

### CROSS-REFERENCE TO RELATED PATENT APPLICATIONS

The present application claims benefit of the filing date of Indian Provisional Patent Application No. 1255/MUM/2008 filed Jun. 13, 2008, which is entirely incorporated herein by reference.

### FIELD OF THE INVENTION

The present invention relates to the preparation of arylcyclopropanecarbonitriles, and compounds derived therefrom, in particular arylcyclopropanecarboxylic acids. The present invention, in particular, provides a process which is cost effective, employs safe water-soluble reagents, in high yields a with purity of at least 99%.

### BACKGROUND OF THE INVENTION

Arylcyclopropanecarbonitriles are useful as intermediates in the preparation of many biologically active compounds. In particular, they are useful in the preparation of arylcyclopropanecarboxylic acids, which have also been used as intermediates, as well as antidotes for herbicide injury to plants.

Conventional methods for the synthesis of arylcyclopropanecarbonitriles and arylcyclopropanecarboxylic acids are described as herein below.

U.S. Pat. No. 4,859,232 describes a procedure wherein phenylacetonitrile derivatives are treated with 1,2-dibromoethane in presence of a phase transfer catalyst in a highly alkaline medium employing 50% NaOH solution. This process involves a highly exothermic reaction in the absence of a solvent, thus posing safety problems.

Further, U.S. Pat. No. 3,721,711 describes a process for preparing 1-(methylthio) cyclopropane carbonitrile wherein a mixture of sodium amide and 1,2-dibromoethane was used to achieve the desired product, and the reaction was carried under inert atmosphere.

EP-A-1666473 illustrates the preparation of methyl 2-(4-chloro-3-nitrophenyl) cyclopropanoate wherein a solution of methyl 2-(4-chloro-3-nitrophenyl)acetate and dibromoethane is reacted in N-methyl-2-pyrolidone (NMP) in presence of sodium hydride to give the desired cyclopropyl derivatives.

EP-A-618900 and U.S. Pat. No. 5,519,034 describe the preparation of cyclopropanecarboxylic acid by using solid KOH as base and 18-Crown-6 as a phase transfer catalyst in DMSO as solvent.

U.S. Published Patent application 20050288338 provides a process for preparation of methyl-1-(phenylthio) cyclopropanecarboxylate by using sodium hydride and DMSO.

U.S. Published Patent application 20050282858 has provided a process for preparation of 1-[4-(difluromethoxy) phenyl] cyclopropane carboxylic acid wherein sodium hydroxide was used as a base in 1-bromo-2-chloro-ethane to yield the cyclopropane carboxylic acid derivative.

Thus, the prior art reveals that the process of the preparation of the arylcyclopropanecarbonitrile and/or carboxylic acid employed various solvents such as

The present invention thus overcomes the above issues by using safe reagents, and has also achieved better yield and pure arylcyclopropane carboxylic acid derivatives.

### OBJECT OF THE INVENTION

It is an object of the present invention to provide efficient process for preparation of arylcyclopropanecarbonitriles and arylcyclopropanecarboxylic acids and their derivatives.

It is an object of the present invention to provide a process for preparation of arylcyclopropane carboxylic acids in high yields of at least 85% with respect to starting material.

It is an object of the present invention to provide a process for the preparation of arylcyclopropane carboxylic acids with purity of at least 99%.

It is an object of the present invention to provide a process with safe operations for the preparation of arylcyclopropanecarboxylic acids.

It is an object of the present invention to provide a process which employs a water miscible solvent.

It is an object of the present invention to provide a process which employs a less hazardous reagent which is also not corrosive.

### SUMMARY OF THE INVENTION

The present invention therefore provides a process for preparing an arylcyclopropane carbonitrile, which process comprises reacting an arylacetonitrile with a compound of formula:
R′ and R″ are the same or different and each represent hydrogen or C₁-C₄ alkyl and R′″ represents phenyl or C₁-C₆ alkyl.

The invention includes the above-noted process in paragraphs [0024] and

wherein the arylacetonitrile of the formula (I) has the formula (Ia),

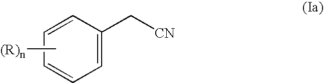

wherein n is 0, 1, 2, 3, 4 or 5 and each R is the same or different and is selected from halogen, C₁-C₆ alkyl, C₁-C₆ alkoxy, hydroxy, C₁-C₆ haloalkyl, C₁-C₆ haloalkoxy, nitro, cyano and —NR′R″, wherein R′ and R″ are the same or different and each independently represent hydrogen or C₁-C₄ alkyl.

In the process according to the invention, the sodium hydroxide powder is added to the reaction medium as the base, and the reaction takes place in the presence of a phase transfer catalyst.

A feature of the invention is that the process set forth in paragraph [0024] also includes:
(a) adding the arylacetonitrile to the sulfolane solvent;
(b) adding sodium hydroxide powder and, optionally, a phase transfer catalyst; and
(c) adding to the thus obtained reaction medium L₁-CH₂—CH₂-L₂, wherein L₁ and L₂ are as defined in claim 1, with stirring at room temperature.

It should be noted that the biologically active compound is a triazole-ethanol fungicide and the biologically active compound is a pesticide.

The biologically active compound fungicide referred to in the paragraph [0029] is an arylcyclopropanecarboxylic acid, or a salt thereof, and process step (b) in paragraph [0028] of reacting the arylcyclopropanecarbonitrile comprises hydrolyzing the arylcyclopropanecarbonitrile in the presence of an acid, to obtain thereby an arylcyclopropanecarboxylic acid.

The invention further consists in a process for preparing an arylcyclopropanemethanol insecticide, which process comprises:
(a) preparing an arylcyclopropanecarboxylic acid by a process according to the process described in paragraph [0028]; and
(b) reducing the thus obtained carboxylic acid to obtain thereby an arylcyclopropanemethanol insecticide.

The invention also consists in the provision of a process for preparing a prostaglandin D₂ antagonist of formula (V), or a pharmaceutically acceptable salt thereof:

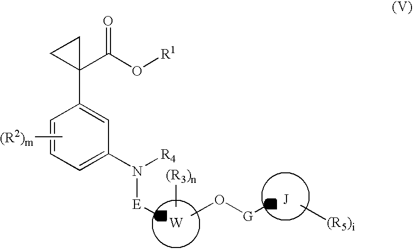

wherein:
R¹ represents a hydrogen atom, C₁-C₄ alkyl, C₂-C₄ alkenyl, or benzyl; E represents —CO‘3, —SO₂—, or —CH₂—;
each R² is the same or different and represents a halogen atom, C₁-C₆ alkyl, C₁-C₆ alkoxy, hydroxyl, trihalomethyl, cyano, phenyl, pyridyl, nitro, C₁-C₄ alkoxy-C₁-C₄ alkyl, phenoxy-C₁-C₄ alkyl, pyridyloxy-C₁-C₄ alkyl, C₁-C₆ hydroxyalkyl, —NR′R″, —SO₂R′″, —SOR″ or —SR′″, wherein each R′ and R″ are the same or different and represent hydrogen or C₁-C₄ alkyl and R′″ represents phenyl or C₁-C₆alkyl;
each R³ is the same or different and represents a halogen atom, C₁-C₆ alkyl, C₁-C₆ alkoxy, hydroxyl, trihalomethyl, cyano, phenyl, pyridyl, nitro, C₁-C₄ alkoxy-C₁-C₄ alkyl, phenoxy-C₁-C₄ alkyl, pyridyloxy-C₁-C₄ alkyl, C₁-C₆ hydroxyalkyl, —NR′R″, —SO₂R′″, —SOR″ or —SR′″, wherein each R′ and R″ are the same or different and represent hydrogen or C₁-C₄ alkyl and R′″ represents phenyl or C₁-C₆ alkyl;
R⁴ represents a hydrogen atom, C₁-C₆ alkyl, benzyl, or C₁-C₆ hydroxyalkyl;
R⁵ represents C₁-C₆ alkyl, C₁-C₁₀ alkoxy, C₁-C₆ alkyl substituted with C₁-C₆ alkoxy, a halogen atom, hydroxyl, trihalomethyl, nitro, phenyl, phenoxy, oxo, C₂-C₆ acyl, cyano, C₁-C₆ hydroxyalkyl, NR′R″, —SO₂R′″, –SOR′″, or —SR′″, wherein R′ and R″ are the same or different and each represent hydrogen or C₁-C₄ alkyl and R′″ represents phenyl or C₁-C₆ alkyl;
W represents a C₅-C₁₂ monocyclic or bicyclic carboxylic ring or a 5- to 12-membered monocyclic or bicyclic heterocycle;
G represents a linking moiety which is a C₁-C₆ alkylene, C₂-C₆ alkenylene or C₂-C₆ alkynylene group interrupted by from 0 to 2 heteroatoms selected from a nitrogen atom, an oxygen atom and a sulphur atom,
J represents a C₅-C₁₂ monocyclic or bicyclic carboxylic ring or a 5- to 12-membered monocyclic or bicyclic heterocycle;
m represents 0 or an integer of 1 to 4;
n represents 0 or an integer of 1 to 4; and
i represents 0 or an integer of 1 to 11,
which process comprises:
   (a) preparing an arylcyclopropane carboxylic acid by a process described in paragraph [0028] which includes the addition of arylacetonitrile to the sulfolane solvent; and
   (b) esterifying and/or alkylating the thus obtained arylcyclopropane carboxylic acid to obtain thereby a compound of formula (V).

### DETAILED DESCRIPTION OF THE INVENTION

The present invention involves the use of sulfolane as a solvent, which is a stable and effective solvent and does not impart impurities to the reaction. Further the sulfolane solvent can be easily recovered for re-use, for example by flash distillation.

The term “aryl” in arylcyclopropanecarbonitrile as used herein refers to any aryl group which can be unsubstituted or substituted, not limited to phenyl and substituted phenyl, which can be substituted at one or more of its substitutable positions with one or more radicals.

The term “TEBA” as used herein refers to triethylbenzylammonium chloride

The term “room temperature” as used herein refers to temperature ranging from 18-25° C.

As used herein, a C₁-C₆ alkyl group or moiety is a linear or branched alkyl group or moiety containing from 1 to 6 carbon atoms, such as a C₁-C₄ alkyl group or moiety, for example methyl, ethyl, n-propyl, i-propyl, i-butyl and t-butyl. A C₁-C₆ alkylene moiety is a said C₁-C₆ alkyl group which is bivalent.

As used herein, a C₂-C₆ alkenyl group or moiety is a linear or branched alkenyl group or moiety containing from 2 to 6 carbon atoms, such as a C₂-C₄ alkenyl group or moiety, for example ethenyl, propenyl and butenyl.

As used herein, a C₂-C₆ alkynyl group or moiety is a linear or branched alkynyl group or moiety containing from 2 to 6 carbon atoms, such as a C₂-C₄ alkynyl group or moiety, for example ethynyl, propynyl and butynyl.

A C₂-C₄ alkenylene or alkynylene group is typically a said alkenyl or alkynyl moiety which is bivalent.

As used herein, a halogen is typically chlorine, fluorine, bromine or iodine and is preferably chlorine or fluorine. As used herein, a said alkoxy group is typically a said alkyl group attached to an oxygen atom.

As used herein, a haloalkyl group is typically a said alkyl group substituted by one or more said halogen atoms. Typically, it is substituted by 1, 2 or 3 said halogen atoms. Preferred haloalkyl groups include perhaloalkyl groups such as —CX₃ wherein X is a said halogen atom. Particularly preferred haloalkyl groups are —CF₃ and —CCl₃.

As used herein, a C₁-C₆ hydroxyalkyl group is a C₁-C₆ alkyl group substituted by one or more, preferably 1, 2 or 3, hydroxy groups. When two hydroxy substituents are present on the same carbon atom, the acetal or ketal moiety will exist in equilibrium with the corresponding ketone or aldehyde moiety in solution, and the C₁-C₆ hydroxyalkyl group can, of course, be used in either form. Preferably, a C₁-C₆ hydroxyalkyl group is substituted by a single hydroxy substituent.

As used herein, a C₂-C₆ aryl group is typically a moiety R—CO—, wherein R is a C₁-C₅ alkyl group.

As used herein, a C₃-C₆ cycloalkyl group is preferably a C₅-C₆ cycloalkyl group.

As used herein, a salt is a salt with any acid or base. Preferred salts are pharmaceutically acceptable salts. These include salts with acids such as inorganic acids, for example hydrochloric, sulphuric, phosphoric, diphosphoric, hydrobromic or nitric acid and organic acids such as citric, fumaric, maleic, malic, ascorbic, succinic, tartaric, benzoic, acetic, methanesulphonic, ethanesulphonic, benzenesulphonic or p-toluenesulphonic acid. Appropriate bases for salt formation include alkali metal (e.g. sodium and potassium) and alkali earth metal (e.g. calcium and magnesium) hydroxides, and organic basis such as alkylamines, arylalkylamines and heterocyclic amines.

The present invention provides a simple and efficient process for the preparation of aryl cyclopropane carbonitriles, wherein it employs a safe reagent such as hydroxides and uses sulpholane as a solvent. These reagents are less hazardous than DMSO, and 50% NaOH.

The present invention thus provides a process for preparation of arylcyclopropanecarbonitrile and its derivatives by employing a safe reagent and less corrosive base.

The present invention employs sulfolane as a solvent for the preparation of corresponding arylcyclopropanecarbonitrile from arylacetonitrile. It is a finding of the present invention that sulfolane provides excellent extraction properties in comparison with other solvents. In addition, sulfolane has the advantage of having good solvent properties such as high density, low heat capacity, and appropriate boiling point, which help simplify separation of the solvent from extract. Hence the present invention has demonstrated that sulfolane can be used as a commercially feasible solvent for preparation of arylcyclopropanecarbonitrile and its derivatives. The solvent sulfolane is typically used in an amount of 2-6 times by volume, and since it is less hazardous than the typically used DMSO, the reaction, is safe to handle.

Typically, therefore, the reaction of the arylacetonitrile with the compound L₁-CH₂—CH₂-L₂ takes place in a solvent which comprises at least 50% sulfolane (vol/vol), preferably at least 60% sulfolane, more preferably at least 75% sulfolane, more preferably at least 90% sulfolane. Co-solvents which can be present in addition to the sulfolane are those known in the art for such nucleophilic substitution reactions, preferably polar aprotic solvents. Preferably, however, DMSO, ethanediol, dimethylsulphate and NMP are not present as co-solvents. Most preferably, the reaction takes place in a single solvent, which is sulfolane.

Typically, L₁ and L₂ in the formula L₁-CH₂—CH₂-L₂ are the same or different and represent halogen, mesylate (CH₃—SO₂—O—), tosylate (4-phenyl-SO₂—O—) or triflate (CF₃—SO₂—O—) groups. Preferably, L₁ and L₂ are the same or different and each represent a halogen atom. More preferably, the reagent L₁-CH₂—CH₂-L₂ is Br—CH₂—CH₂—Br.

Typically, the arylacetonitrile has the formula (I)

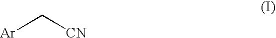

wherein Ar is a phenyl or naphthyl group, which is unsubstituted or carries one or more substituent selected from halogen, C₁-C₆ alkyl, C₁-C₆ alkoxy, hydroxy, C₁-C₆ haloalkyl, C₁-C₆ haloalkoxy, (C₁-C₆ alkyl)oxycarbonyl, —CO₂R′, nitro, cyano, cyano-(C₃-C₆ cycloalkyl)-, phenyl, phenoxy, pyridyl, pyridyloxy, (C₁ -C₄ alkoxy)-(C₁-C₄ alkyl)-, phenoxy-C₁-C₄ alkyl-, pyridyloxy-C₁-C₄ alkyl-, —NR′R″, C₁-C₆ hydroxyalkyl, SO₂R′″, —SOR′″ or SR′″, wherein each R′ and R″ are the same or different and each represent hydrogen or C₁-C₄ alkyl and R′″ represents phenyl or C₁-C₆ alkyl.

Typically, only 1 or 2 of the substituents on the group Ar are selected from (C₁-C₆ alkyl)oxycarbonyl, —CO₂—R′, nitro, cyano-(C₃-C₆ cycloalkyl)-, phenyl, phenoxy, pyridyl, pyridyloxy, phenoxy-(C₁-C₄ alkyl), pyridyloxy-(C₁-C₄ alkyl)-, C₁-C₆ hydroxyalkyl, —SO₂R′″, —SOR′″ and —SR′″ substituents. Typically, when Ar is naphthyl it is unsubstituted. Preferably, Ar is phenyl.

Preferably, the substituents on Ar are selected from halogen, C₁-C₆ alkyl, C₁-C₆ alkoxy, hydroxy, C₁-C₆ haloalkyl, C₁-C₆ haloalkoxy, nitro, cyano and —NR′R″, wherein R′ and R″ are the same or different and each independently represent hydrogen or C₁-C₄ alkyl.

More preferably, the arylacetonitrile of the formula (I) has the formula (Ia),

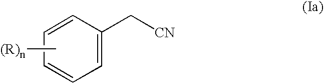

wherein n is 0, 1, 2, 3, 4 or 5 and each R is the same or different and is selected from halogen, C₁-C₆ alkyl, C₁-C₆ alkoxy, hydroxy, C₁-C₆ haloalkyl, C₁-C₆ haloalkoxy, nitro, cyano and —NR′R″, wherein R′ and R″ are the same or different and each independently represent hydrogen or C₁-C₄ alkyl.

The use of sulfolane as a solvent enables the use, if desired, of a less corrosive base than the aqueous NaOH previously employed in such reactions. Thus, typically, the base present in the process of the invention is sodium hydroxide powder.

Typically the process of the present invention for preparing arylcyclopropanecarbonitriles from arylacetonitriles is carried out by stirring the arylacetonitrile compound in sulfolane solvent, and adding sodium hydroxide powder and a phase transfer catalyst such as TEBA at room temperature. The reactant L₁-CH₂—CH₂-L₂ is then typically added under stirring at room temperature. The temperature of the reaction mixture can then be slowly raised. The progress of the reaction can be readily monitored by techniques such as TLC. After completion of the reaction the reaction mass can be quenched into water and the layers can be separated. The product in the aqueous layer can be further extracted with any suitable solvent. The sulfolane solvent can be removed by distillation and the residue can be purified by fractional distillation, or recrystallisation.

As noted above, arylcyclopropanecarbonitriles are useful as intermediates in the preparation of many biologically active compounds. The present invention therefore also provides a process for preparing a biologically active compound, which process comprises:
(a) preparing an arylcyclopropanecarbonitrile by a process according to the invention; and
(b) further reacting the thus obtained arylcyclopropanecarbonitrile to obtain a biologically active compound.

Step (b) above may comprise one or more reaction steps. In particular, it may comprise:
(i) acid hydrolysis, to obtain a corresponding arylcyclopropanecarboxylic acid; and
(ii) further reaction steps, to yield a biologically active compound.

A “biologically active compound” is typically a pharmaceutical, a herbicide, a pesticide or an insecticide.

Typically, in one embodiment, said biologically active compound is a fungicide and an anti-mycotic. In this embodiment, said biologically active compound is typically a triazole ethanol compound as described in Belgian patent no. 900,594 and GB-A-2146987. In this embodiment, the arylacetonitrile of the formula (I) typically has the formula (Ia), wherein n is 1 and R is halogen. Typically, in this embodiment, the arylcyclopropane carbonitrile is 1-(2-fluorophenyl)cyclopropanecarbonitrile, 1-(4-fluorophenyl)cyclopropanecarbonitrile or 1-(4-chlorophenyl)cyclopropanecarbonitrile.

Typically, in this embodiment, the biologically active compound has the formula:

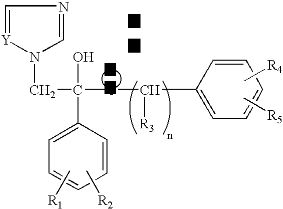

wherein
R₁ and R₂, independently, are hydrogen, halogen, nitro, C₁-C₅ alkyl, C₁-C₅ haloalkyl, C₂-C₅ alkenyl, C₂-C₅ alkynyl, C₁-C₅ alkoxy or C₁-C₅ alkylthio or unsubstituted or substituted phenyl or phenoxy,
R₃ is hydrogen or C₁-C₅ alkyl,
R₄ and R₅, independently, are hydrogen or halogen,
Y is CH or N,
A is an ethylene bridge, and
n is 0,
   in free base form or in the form of an acid addition salt or a physiologically-hydrolysable and acceptable derivative.

Preferred substituents on a “substituted” phenyl or phenoxy group are those set out above as substituents on Ar.

In this embodiment, particularly preferred biologically active compounds are:
1-[1(4-chlorophenyl)-1-hydroxy-2-(1H-1,2,4-triazol-1-yl)ethyl]-1-(4-chlorophenyl)cyclopropane;
1-[1(4-chlorophenyl)-1-hydroxy-2-(1H-1,3-imidazol-1-yl)ethyl]-1-(4-chlorophenyl)cyclopropane;
1-[1(4-chlorophenyl)-1-hydroxy-2-(1H-1,2,4-triazol-1-yl)ethyl]-1-(2-fluorophenyl)cyclopropane;
1-[1(4-fluorophenyl)-1-hydroxy-2-(1H-1,2,4-triazol-1-yl)ethyl]-1-(4-fluorophenyl)cyclopropane; and
1-[1(4-chlorophenyl)-1-hydroxy-2-(1H-1,2,4-triazol-1-yl)ethyl]-1-phenyl-cyclopropane.

In a further embodiment, the biologically active compound is a pesticide as disclosed in Belgian patent no. 902,147 and WO 85/04651. In this embodiment, the arylacetonitrile of the formula (I) typically has the formula (Ia), wherein n is 1 and R is C₁-C₄ alkoxy. Typically, the cyclopropanecarbonitrile is 1-(4-ethoxyphenyl)cyclcopropanecarbonitrile.

Preferably, the biologically active compound is an arylcyclopropanecarboxlic acid. Thus, the present invention further provides a process for preparing an arylcyclopropylcarboxylic acid, or a salt thereof, which process comprises:
(a) preparing an arylcyclopropanecarbonitrile by a process according to the invention; and
(b) hydrolysing the thus obtained arylcyclopropanecarbonitrile in the presence of an acid, to obtain thereby an arylcyclopropylcarboxylic acid.

In this preferred embodiment the present invention provides a process, for example as per scheme I shown in the Examples, wherein substituted arylacetonitrile is treated with dihaloethane (for example) in presence of alkali and sulpholane and the resulting arylcyclopropane carbonitrile is hydrolysed with sulphuric acid to give arylcarboxylic acids.

In this embodiment the present invention can provide a process for preparation of arylcyclopropanecarboxylic acids with yields of at least 85% with respect to the arylacetonitriles as a starting material.

In this embodiment, further, the present invention can provide a process for the preparation of arylcyclopropane carboxylic acids with high purity of at least 99%, using water-soluble reagents that can be easily removed, thus rendering highly pure compounds. Thus, the present invention can provide a simple process for preparing aryl carboxylic acids that can be scaled up on a commercial level.

Typically, in this embodiment, in step (b) the arylcyclopropanecarbonitrile is converted to its corresponding acid by acidic hydrolysis. Typically, hydrolysis is effected with 20% sulfuric acid. Typically, the hydrolysis is carried out at higher temperature such as 100-101° C. for about 12 hours. After completion of the reaction a suitable solvent such as ethylacetate can be used for extracting the product. The acid product can be purified by extraction into aqueous alkali and reprecipitating the product at acidic pH with conc. HCl. The pure product can then be filtered and washed with water and dried.

Preferably, in this embodiment, the aryl-cyclopropane carboxylic acid is one of the compounds exemplified as “antidote compounds 1 to 43” in U.S. Pat. No. 4,859,232.

The arylcyclopropyl carboxylic acids discussed above are themselves useful as intermediates in the preparation of further biologically active compounds. In particular, they are useful, for example, in the preparation of insecticides and pharmaceuticals.

The present invention therefore also provides a process for preparing a process for preparing an arylcyclopropanemethanol insecticide, which process comprises:
(a) preparing an arylcyclopropane carboxylic acid by a process according to the invention; and
(b) reducing the thus obtained carboxylic acid to obtain thereby an arylcyclopropane methanol insecticide.

Preferably, in this embodiment, the insecticide is 1-(4-chlorophenyl)cyclopropanemethanol as described in EP-A-0094085.

Also provided is a process for preparing a process for preparing a prostaglandin D₂ antagonist of formula (V), or a pharmaceutically acceptable salt thereof:

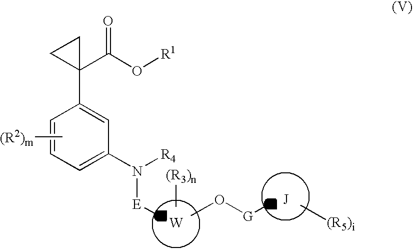

wherein:
R¹ represents a hydrogen atom, C₁-C₄ alkyl, C₂-C₄ alkenyl, or benzyl;
E represents —CO—, —SO₂—, or —CH₂—;
each R² is the same or different and represents a halogen atom, C₁-C₆ alkyl, C₁-C₆ alkoxy, hydroxyl, trihalomethyl, cyano, phenyl, pyridyl, nitro, C₁-C₄ alkoxy-C₁-C₄ alkyl, phenoxy-C₁-C₄ alkyl, pyridyloxy-C₁-C₄ alkyl, C₁-C₆ hydroxyalkyl, —NR′R″, —SO₂R′″, —SOR″ or —SR′″, wherein each R′ and R″ are the same or different and represent hydrogen or C₁-C₄ alkyl and R′″ represents phenyl or C₁-C₆ alkyl;
each R³ is the same or different and represents a halogen atom, C₁-C₆ alkyl, C₁-C₆ alkoxy, hydroxyl, trihalomethyl, cyano, phenyl, pyridyl, nitro, C₁-C₄ alkoxy-C₁-C₄ alkyl, phenoxy-C₁-C₄ alkyl, pyridyloxy-C₁-C₄ alkyl, C₁-C₆ hydroxyalkyl, —NR′R″, —SO₂R′″, —SOR″ or —SR′″, wherein each R′ and R″ are the same or different and represent hydrogen or C₁-C₄ alkyl and R′″ represents phenyl or C₁-C₆ alkyl;
R⁴ represents a hydrogen atom, C₁-C₆ alkyl, benzyl, or C₁-C₆ hydroxyalkyl;
R⁵ represents C₁-C₆ alkyl, C₁-C₁₀ alkoxy, C₁-C₆ alkyl substituted with C₁-C₆ alkoxy,
a halogen atom, hydroxyl, trihalomethyl, nitro, phenyl, phenoxy, oxo, C₂-C₆ acyl, cyano, C₁-C₆ hydroxyalkyl, NR′R″, —SO₂R′″, –SOR′″, or —SR′″, wherein R′ and R″ are the same or different and each represent hydrogen or C₁-C₄ alkyl and R′″ represents phenyl or C₁-C₆ alkyl;
W represents a C₅-C₁₂ monocyclic or bicyclic carboxylic ring or a 5- to 12-membered monocyclic or bicyclic heterocycle;
G represents a linking moiety which is a C₁-C₆ alkylene, C₂-C₆ alkenylene or C₂-C₆
alkynylene group interrupted by from 0 to 2 heteroatoms selected from a nitrogen atom, an oxygen atom and a sulphur atom;
J represents a C₅-C₁₂ monocyclic or bicyclic carboxylic ring or a 5- to 12-membered monocyclic or bicyclic heterocycle;
m represents 0 or an integer of 1 to 4;
n represents 0 or an integer of 1 to 4; and
i represents 0 or an integer of 1 to 11.
which process comprises:
   (a) preparing an arylcyclopropane carboxylic acid by a process according the invention; and
   (b) esterifying and/or alkylating the thus obtained arylcyclopropane carboxylic acid to obtain thereby a compound of formula (V).

In this embodiment, the compounds of formula (V) are those set out in formula (I) disclosed in EP-A-1666473. Preferred meanings for the substituent definitions in the formula (V) are those set out in EP-A-1666473, and preferred compounds of formula (V) are those exemplified in EP-A-1666473. In particular, preferred C₅-Cl₁₂ monocyclic and bicyclic carboxylic rings and preferred 5- to 12-membered monocyclic and bicyclic heterocycles are those set out at paragraphs [0031] and [0032] of EP-A-1666473.

It should be appreciated that the specific reaction parameters (e.g. temperature and time taken for the reaction) for the process steps described above can be readily optimized by any skilled person in the art for a particular product. The present invention thus provides a commercially feasible process by employing sulfolane as a solvent thus reducing the environmental hazards and also provides a process wherein a less corrosive base is used.

The following Examples are included to demonstrate preferred embodiments of the invention. It should be appreciated by those of skill in the art that the techniques disclosed in the Examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

All parts are by weight unless otherwise indicated. Most of the commercially-available starting materials were obtained from Aldrich Chemical Co.

### EXAMPLE 1

### Preparation of para-chlorocyclopropanecarbonitrile

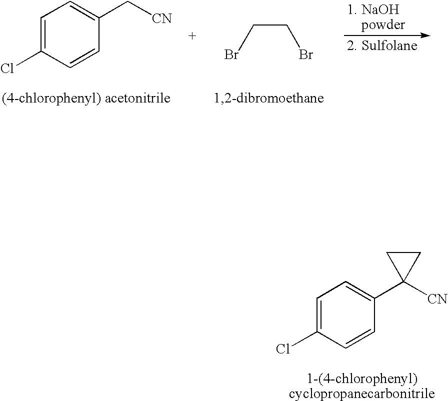

To a solution of Sulfolane (250 ml), p-chlorophenylacetonitrile (100 gm) was added under stirring. Sodium hydroxide powder (13.19 gm, 5.0 mole) and TEBAC (0.05% )751 gm was added to the reaction mixture and stirred for 10-40 minutes.

Then 1,2-dibromoethane (37.17 gm, 3.0 mole) was added to the reaction mass and the temperature of the reaction was slowly raised and maintained at 50-70° C. for 14-16 hours. The progress of the reaction was monitored by Thin Layer chromatography (Solvent system). After 14-16 hours, the reaction mass was quenched into water (˜600ml) and the organic layer was separated. The aqueous layer was washed with ethyl acetate. The organic layer was distilled under vacuum to remove the solvent and then the residue was subjected to fractional distillation at 2mm Hg vacuum at 120-130° C. to yield p-chlorocyclopropanecarbonitrile product (85-90%).

The purity of the product was 99.0% by HPLC and the Melting point: 140-143° C.

### EXAMPLE 2

### Preparation of p-chlorophenycyclopropane carboxylic acid

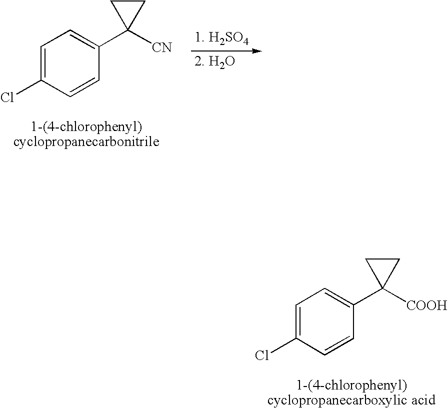

The cyclopropanecarbonitrile (18 gm) obtained in example 1 was added to 20% sulfuric acid (184 ml). The temperature of the reaction mass was slowly raised and maintained at reflux for 10-12 hours. After 12 hours, ethyl acetate was used to extract the product. The acid product from the organic layer was then extracted into 20% NaOH solution. The aqueous layer was then acidified to pH 2-4 with concentrated HCl. The white solid product obtained was filtered and washed with water. The product p-chlorophenylcyclopropanecarboxylic acid was dried. The yield was 90-95% and the product had a purity of 99.4% by HPLC. The melting point was 152-155 ° C.

All of the methods disclosed and claimed herein can be made and executed without undue experimentation in light of the present disclosure. While the methods of this invention have been described in terms of preferred embodiments, it will be apparent to those of skill in the art that variations may be applied to the compositions and/or methods and in the steps or in the sequence of steps of the methods described herein without departing from the concept, spirit and scope of the invention. More specifically, it will be apparent that certain agents that are chemically or physiologically related may be substituted for the agents described herein while the same or similar results would be achieved. All such similar substitutes and modifications apparent to those skilled in the art are deemed to be within the spirit, scope and concept of the invention.

1. A process for preparing an arylcyclopropanecarbonitrile, or a salt thereof, which process comprises reacting an arylacetonitrile with a compound of formula L₁-CH₂—CH₂-L₂, wherein L₁ and L₂ are the same or different and each represent a leaving group, in a sulfolane solvent, in the presence of a base.

2. A process according to claim 1, wherein the arylacetonitrile has the formula (I) 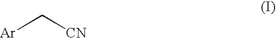
wherein Ar is a phenyl or naphthyl group, which is unsubstituted or carries one or more substituent selected from halogen, C₁-C₆ alkyl, C₁-C₆ alkoxy, hydroxy, C₁-C₆ haloalkyl, C₁-C₆ haloalkoxy, (C₁-C₆ alkyl)oxycarbonyl, —CO₂R′, nitro, cyano, cyano-(C₃-C₆ cycloalkyl), phenyl, phenoxy, pyridyl, pyridyloxy, (C₁-C₄ alkoxy)-(C₁-C₄ alkyl)-, phenoxy-C₁-C₄ alkyl-, pyridyloxy-C₁-C₄ alkyl-, —NR′R″, C₁-C₆ hydroxyalkyl, SO₂R′″, —SOR′″ or SR′″, wherein each R′ and R″ are the same or different and each represent hydrogen or C₁-C₄ alkyl and R′″ represents phenyl or C₁-C₆ alkyl.

3. A process according to claim 1, wherein the arylacetonitrile of the formula (I) has the formula (Ia), 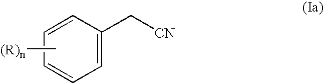
wherein n is 0, 1, 2, 3, 4 or 5 and each R is the same or different and is selected from halogen, C₁-C₆ alkyl, C₁-C₆ alkoxy, hydroxy, C₁-C₆ haloalkyl, C₁-C₆ haloalkoxy, nitro, cyano and —NR′R″; wherein R′ and R″ are the same or different and each independently represent hydrogen or C₁-C₄ alkyl.

4. A process according to claim 2, wherein the arylacetonitrile of the formula (I) has the formula (Ia), 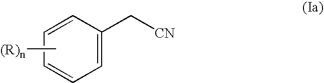
wherein n is 0, 1, 2, 3, 4 or 5 and each R is the same or different and is selected from halogen, C₁-C₆ alkyl, C₁-C₆ alkoxy, hydroxy, C₁-C₆ haloalkyl, C₁-C₆ haloalkoxy, nitro, cyano and —NR′R″, wherein R′ and R″ are the same or different and each independently represent hydrogen or C₁-C₄ alkyl.

5. A process according to claim 1 wherein sodium hydroxide powder is added to the reaction medium as the base.

6. A process according to claim 1, wherein the reaction takes place in the presence of a phase transfer catalyst.

7. A process according to claim 2, wherein sodium hydroxide powder is added to the reaction medium as the base.

8. A process according to claim 2, wherein the reaction takes place in the presence of a phase transfer catalyst.

9. A process according to claim 3, wherein sodium hydroxide powder is added to the reaction medium as the base.

10. A process according to claim 3, wherein the reaction takes place in the presence of a phase transfer catalyst.

11. A process according claim 1, which comprises:
(a) adding the arylacetonitrile to the sulfolane solvent;
(b) adding sodium hydroxide powder and, optionally, a phase transfer catalyst; and
(c) adding to the thus obtained reaction medium L₁-CH₂—CH₂-L₂, wherein L₁ and L₂ are as defined in claim 1, with stirring at room temperature.

12. A process according to claim 2, which comprises:
(a) adding the arylacetonitrile to the sulfolane solvent;
(b) adding sodium hydroxide powder and, optionally, a phase transfer catalyst; and
(c) adding to the thus obtained reaction medium L₁-CH₂—CH₂-L₂ are as defined in claim 1, with stirring at room temperature.

13. A process according to claim 3, which comprises:
(a) adding the arylacetonitrile to the sulfolane solvent;
(b) adding sodium hydroxide powder and, optionally, a phase transfer catalyst; and
(c) adding to the thus obtained reaction medium L₁-CH₂—CH₂-L₂, wherein L₁ and L₂ are as defined in claim 1, with stirring at room temperature.

14. A process for preparing a biologically active compound, which process comprises:
(a) preparing an arylcyclopropanecarbonitrile by a process according to claim 1; and
(b) further reacting the thus obtained arylcyclopropanecarbonitrile to obtain a biologically active compound.

15. A process according to claim 14, wherein the biologically active compound is a triazole-ethanol fungicide.

16. A process according to claim 14, wherein the biologically active compound is a pesticide.

17. A process according to claim 14, wherein the biologically active compound is an arylcyclopropanecarboxylic acid, or a salt thereof, and process step (b) comprises hydrolyzing the arylcyclopropanecarbonitrile in the presence of an acid, to obtain thereby an arylcyclopropanecarboxylic acid.

18. A process for preparing an arylcyclopropanemethanol insecticide, which process comprises:
(a) preparing an arylcyclopropanecarboxylic acid by a process according to claim 11; and
(b) reducing the thus obtained carboxylic acid to obtain thereby an arylcyclopropanemethanol insecticide.

19. A process for preparing a prostaglandin D₂ antagonist of formula (V), or a pharmaceutically acceptable salt thereof: 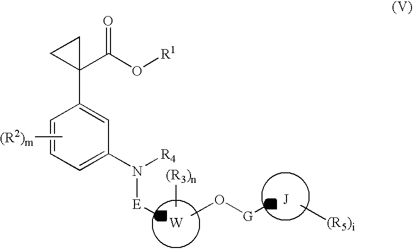
wherein:
R¹ represents a hydrogen atom, C₁-C₄ alkyl, C₂-C₄ alkenyl, or benzyl;
E represents —CO—, —SO₂—, or —CH₂—;
each R² is the same or different and represents a halogen atom, C₁-C₆ alkyl, C₁-C₆ alkoxy, hydroxyl, trihalomethyl, cyano, phenyl, pyridyl, nitro, C₁-C₄ alkoxy-C₁-C₄ alkyl, phenoxy-C₁-C₄ alkyl, pyridyloxy-C₁-C₄ alkyl, C₁-C₆ hydroxyalkyl, —NR′R″, —SO₂R′″, —SOR″ or —SR′″, wherein each R′ and R″ are the same or different and represent hydrogen or C₁-C₄ alkyl and R′″ represents phenyl or C₁-C₆ alkyl;
each R³ is the same or different and represents a halogen atom, C₁-C₆ alkyl, C₁-C₆ alkoxy, hydroxyl, trihalomethyl, cyano, phenyl, pyridyl, nitro, C₁-C₄ alkoxy-C₁-C₄ alkyl, phenoxy-C₁-C₄ alkyl, pyridyloxy-C₁-C₄ alkyl, C₁-C₆ hydroxyalkyl, —NR′R″, —SO₂R′″, —SOR″ or —SR′″, wherein each R′ and R″ are the same or different and represent hydrogen or C₁-C₄ alkyl and R′″ represents phenyl or C₁-C₆ alkyl;
R⁴ represents a hydrogen atom, C₁-C₆ alkyl, benzyl, or C₁-C₆ hydroxyalkyl;
R⁵ represents C₁-C₆ alkyl, C₁ -C₁₀ alkoxy, C₁-C₆ alkyl substituted with C₁-C₆ alkoxy, a halogen atom, hydroxyl, trihalomethyl, nitro, phenyl, phenoxy, oxo, C₂-C₆ acyl, cyano, C₁-C₆ hydroxyalkyl, NR′R″, —SO₂R′″, —SOR′″, or –SR′″, wherein R′ and R″ are the same or different and each represent hydrogen or C₁-C₄ alkyl and R′″ represents phenyl or C₁-C₆ alkyl;
W represents a C₅-C₁₂ monocyclic or bicyclic carboxylic ring or a 5- to 12-membered monocyclic or bicyclic heterocycle;
G represents a linking moiety which is a C₁-C₆ alkylene, C₂-C₆ alkenylene or C₂-C₆ alkynylene group interrupted by from 0 to 2 heteroatoms selected from a nitrogen atom, an oxygen atom and a sulphur atom;
J represents a C₅-C₁₂ monocyclic or bicyclic carboxylic ring or a 5- to 12-membered monocyclic or bicyclic heterocycle;
m represents 0 or an integer of 1 to 4;
n represents 0 or an integer of 1 to 4; and
i represents 0 or an integer of 1 to 11,
which process comprises:
(a) the process commencing with the addition of arylacetonitrile; and
(b) esterifying and/or alkylating the thus obtained arylcyclopropane carboxylic acid to obtain thereby a compound of formula (V).

20. A process according to claim 4, which comprises:
(a) adding the arylacetonitrile to the sulfolane solvent;
(b) adding sodium hydroxide powder and, optionally, a phase transfer catalyst; and
(c) adding to the thus obtained reaction medium L₁ -CH₂—CH₂-L₂,. Wherein L₁ and L₂ are as defined in claim 1, with stirring at room temperature.
